(19) United States
(12) Patent Application Publication
(10) Pub. No.: US 2010/0256100 A1
(43) Pub. Date: Oct. 7, 2010

(54) ROLL-ON WITH HIGH SALICYLIC ACID CONTENT

(75) Inventors: Sylvie Quedville, Gennevillers (FR); Geoffroy Remaut, Fresnes (FR); Anthony Potin, Montrouge (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'OREAL, Paris (FR)

(21) Appl. No.: 12/751,483

(22) Filed: Mar. 31, 2010

Related U.S. Application Data
(60) Provisional application No. 61/168,231, filed on Apr. 10, 2009.

(30) Foreign Application Priority Data
Apr. 1, 2009 (FR) 09 52105

(51) Int. Cl. A61K 31/60; A61P 17/10; A45D 34/04

(52) U.S. Cl. 514/159; 401/209

(57) ABSTRACT

The present invention relates to an assembly (1) for packaging and application of a cosmetic and/or dermatological composition, comprising a container (2) having an opening (3), an applicator element (4), mounted in such a way as to be freely rotatable, in the vicinity of said opening (3), the applicator element (4) comprising an outer surface which can be brought into fluidic communication with the inside of the container (2), characterized in that the container (2) comprises a cosmetic and/or dermatological composition in the form of a gel having a viscosity at 25° C. of between 0.1 and 60 poises, and comprising, in a physiologically acceptable medium:
(a) at least 1% by weight of salicylic acid and/or at least one derivative thereof, relative to the total weight of the composition;
(b) at least one polyol; and
(c) at least one gelling agent.

### REFERENCE TO PRIOR APPLICATIONS

This application claims priority to U.S. provisional application 61/168,231, filed Apr. 10, 2009, and to French patent application 0952105 filed Apr. 1, 2009, both incorporated herein by reference.

### FIELD OF THE INVENTION

The present invention pertains to a packaging and application assembly featuring a roll-on applicator structure, comprising a cosmetic and/or dermatological composition containing a high level of salicylic acid and/or at least one derivative thereof, and also to its use particularly for caring for greasy and/or acne-affected and/or seborrhoeic skin.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Greasy or hyperseborrhoeic skin is characterized by a skin which is shiny, sometimes with an oily appearance, thick, with dilated pilosebaceous pores. Sebaceous hypersecretion is most commonly associated with hyperandrogenism due either to hyperproduction of androgens by an endocrine gland, or to peripheral hyperproduction at the sebaceous gland on the basis of surrounding androgens and/or proandrogens.

This sebaceous hypersecretion, in combination with an accumulation of epithelial cells resulting from abnormal cell multiplication, may lead to blocking of the sebaceous follicles, which are located in particular on the face, and may progress to comedones.

Moreover, a resident anaerobic bacterium, Propionibacterium acnes, proliferates in this environment rich in sebum and follicular cells, and may result locally in inflammation.

Acne-prone greasy skin therefore generally exhibits cutaneous imperfections, dilated pores, an inhomogeneous texture to the skin, and redness.

There are numerous cosmetic compositions containing salicylic acid for the care and/or treatment of greasy skin. These compositions are generally applied directly by finger or directly by hand to the surface of the skin in need of care.

Furthermore, roll-on devices using an applicator ball are in widespread use for the packaging and application of body deodorants.

Examples of cosmetic and/or hygiene products of roll-on type include deodorants and the Caffeine Eye Roll-On product sold by Garnier in its Nutritionist range.

Other examples of assemblies for packaging and application of a cosmetic product, of roll-on type, are described in patents U.S. Pat. No. 5,553,957, U.S. Pat. No. 4,033,700, U.S. Pat. No. 4,021,125 and U.S. Pat. No. 6,132,126.

In formulating a composition in the form of a gel with a high level of salicylic acid (content greater than or equal to 1%, relative to the total weight of the composition) in an assembly for packaging an application of a cosmetic and/or dermatological composition of roll-on type, the inventors were confronted with the problem of the recrystallization of the salicylic acid on the surface of the applicator element, more particularly a ball.

The reason was that, over time, the applicator ball had a tendency to become blocked as a consequence of surface recrystallization of the salicylic acid, thereby reducing or even negating the functionality of the tool (roll-on), and was therefore detrimental to the quality of the care provided to the users (difficulty of release of the product).

### SUMMARY OF THE INVENTION

The Applicant has found, entirely surprisingly and unexpectedly, that the incorporation of at least one polyol into the composition in the form of a gel with a high level of salicylic acid allows the problem set out above to be addressed.

Advantageously, the incorporation of at least one polyol into the composition in the form of a gel, in particular an aqueous-alcoholic gel, with a high level of salicylic acid, packaged in a packaging and application assembly according to the invention, allows said gel to be stabilized. Stabilizing the gel thus allows avoidance of the phenomena of recrystallization on the surface of the applicator ball following application (drying).

Another advantage of a composition according to the invention packaged in a packaging and application assembly according to the invention is the ability for precise targeting of the area to be treated (for example, the top of the shoulder), resulting in optimum use and local use on the surface of the skin. It is also advantageous to use a packaging assembly as described in the present invention for the care of greasy and/or acne-affected and/or seborrhoeic skin, since the assembly provides a freshness effect (highly desired in the care of these skin types) at the time the composition is applied.

### BRIEF DESCRIPTION OF THE DRAWING

FIG. 1 depicts an assembly for packaging and application of a cosmetic and/or dermatological composition of the present invention.

### DETAILED DESCRIPTION OF THE INVENTION

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. The term “mentioned” notes exemplary embodiments, and is not limiting to certain species. As used herein the words “a” and “an” and the like carry the meaning of “one or more.”

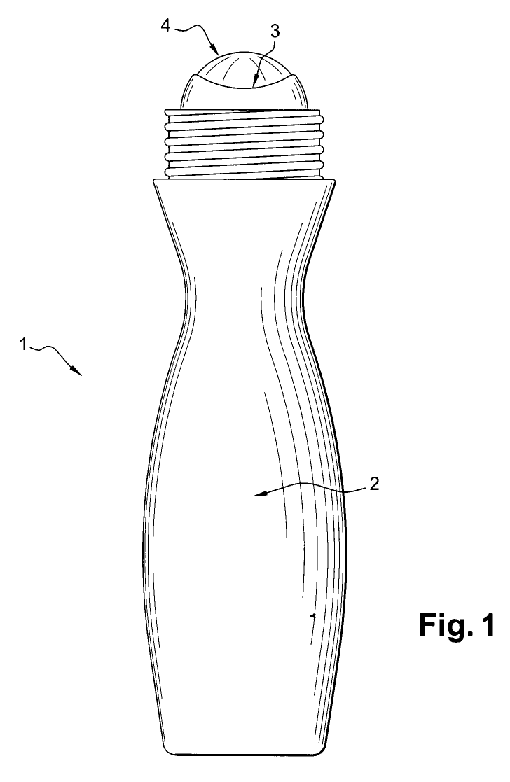

As exemplified in FIG. 1, the present invention principally provides an assembly (1) for packaging and application of a cosmetic and/or dermatological composition, comprising a container (2) having an opening (3), an applicator element (4), mounted in such a way as to be freely rotatable, in the vicinity of said opening (3), the applicator element (4) comprising an outer surface which can be brought into fluidic communication with the inside of the container (2), characterized in that the container (2) comprises a cosmetic and/or dermatological composition in the form of a gel having a viscosity at 25° C. of between 0.1 and 60 poises, and comprising, in a physiologically acceptable medium:
(a) at least 1% by weight of salicylic acid and/or at least one derivative thereof, relative to the total weight of the composition;
(b) at least one polyol; and
(c) at least one gelling agent.

In particular, the applicator element (4), in particular a ball, is mounted, in such a way as to be freely rotatable on itself, in the vicinity of the opening (3) in a correspondingly shaped housing, which sits atop the container containing said composition according to the invention, said housing being in fluidic communication with the inside of the container.

In further detail, the assembly for packaging and application of a cosmetic and/or dermatological composition according to the invention comprises a container (2) having a base and, opposite the base, an opening (3). The housing is defined by a ball support, which may be obtained by monobloc moulding with the container, or may be formed by an insert which attaches on the opening of the container.

In one preferred embodiment, the applicator element is a ball.

In particular, part of the surface of the ball is opposite the opening in the container, so as to allow the product to be applied by the surface of the ball that emerges from the container in the course of its rotational movement on the surface of the skin.

The container may be formed of at least one thermoplastic material such as, for example, polyolefins, more particularly polypropylene, high-density or low-density polyethylene, or mixtures thereof, or, for example, acrylonitrile-butadiene-styrene (ABS).

Other materials may, however, be used.

In one preferred embodiment, the container is formed of at least one thermoplastic material selected from polyolefins, more particularly polypropylene, high-density or low-density polyethylene, or mixtures thereof.

The applicator element may be made of a thermo-plastic material or of metal.

Among thermoplastic materials, consideration may be given to acrylonitrile-butadiene-styrene (ABS) and also to polyolefins, more particularly polypropylene, high-density or low-density polyethylene, or mixtures thereof.

Among metals, mention may be made, for example, of aluminium, stainless steel and silver.

Other materials may, however, be used.

In one preferred embodiment, the applicator element is preferably a ball, more preferably a metal ball, and very preferably a stainless steel ball.

When the applicator element is a ball, the ball may be spherical or oval. With preference it will be spherical.

The dimensions or diameter of the ball will depend, of course, on the size of the opening in the container.

The assembly for packaging and application of a cosmetic and/or dermatological composition according to the invention preferably comprises a removable cap (lid).

It is quite clear that other variants may be applied to the packaging and application assembly without departing from the spirit of the invention as claimed.

Illustratively, FIG. 1 is a collective view (facing view) of a device according to the invention.

FIG. 1 shows an assembly (1) for packaging and application of a cosmetic and/or dermatological composition, comprising a container (2) having an opening (3), and an applicator element (4).

The composition according to the invention is preferably a cosmetic composition. It can also be a dermatological or pharmaceutical composition.

According to the present invention, the cosmetic and/or dermatological composition is in the form of a gel having a viscosity at 25° C. of between 0.1 and 60 poises (between 0.01 Pa.s and 6 Pa.s).

A viscosity of this kind allows the product (composition) to be delivered to the surface of the skin in need of care. In one preferred embodiment, the cosmetic and/or dermatological composition is in the form of a gel having a viscosity at 25° C. of between 0.5 and 10 poises (between 0.05 Pa.s and 1 Pa.s).

Viscosity Measurement

The viscosity of the composition is measured at 25° C. using a Rhéomat 180 (Lamy) fitted with an MS-R1, MS-R2, MS-R, MS-R4 or MS-R5 spindle, selected according to the consistency of the composition, which rotates at a speed of 200 rpm. The measurement is taken after rotation for 10 minutes. The viscosity measurements are carried out no later than 1 week after manufacture.

The composition according to the invention is suitable for topical application to the skin and therefore generally comprises a physiologically acceptable medium, by which is meant a medium compatible with the skin and/or its epidermal derivatives. This is preferably a cosmetically acceptable medium, which in other words has a colour, odour and feel that are pleasant, and does not give rise to unacceptable discomfort (stinging, pulling, redness) that might dissuade a user from employing this composition.

Salicylic Acid and/or Derivatives Thereof:

A composition according to the invention comprises at least 1% by weight of salicylic acid and/or at least one derivative thereof, relative to the total weight of the composition. Thus, by “high level of salicylic acid and/or at least one derivative thereof” is meant an amount of at least 1% (greater than or equal to 1%) by weight of salicylic acid and/or of at least one derivative thereof, relative to the total weight of the composition.

In one preferred embodiment of the invention, the composition preferably comprises salicylic acid.

Salicylic acid is sold in particular by Rhodia under the name Salicylic Acid Pharmaceutical Grade USP, Ph. Eur.

Salicylic acid derivatives include in particular its alkyl derivatives, by which are meant its derivatives of formula (I):

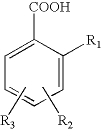

in which:

R₁ represents a hydroxyl radical or an ester radical of formula:

—O—CO—R₄

in which R₄ is a saturated or unsaturated aliphatic radical comprising from 1 to 26 carbon atoms, and preferably from 1 to 18 carbon atoms, or an amine or thiol function which is optionally substituted by an alkyl radical comprising from 1 to 18 carbon atoms, and preferably from 1 to 12 carbon atoms,

R₂ and R₃, independently of one another, are located in position 3, 4, 5 or 6 on the benzene ring and represent, independently of one another, a hydrogen atom or a radical:

(O)ₙ—(CO)ₘ—R₅

in which n and m, independently of one another, are each an integer 0 or 1, with the proviso that R₂ and R₃ are not simultaneously hydrogen atoms;

R₅ represents a hydrogen, a linear, branched or cyclized, saturated aliphatic radical comprising from 1 to 18 carbon atoms, an unsaturated radical comprising from 3 to 18 carbon atoms and bearing one to nine conjugated or non-conjugated double bonds, it being possible for the radicals to be substituted by at least one substituent selected from halogen atoms (fluorine, chlorine, bromine, iodine), trifluoromethyl radicals, hydroxyl radicals in free form or esterified with an acid comprising from 1 to 6 carbon atoms, or carboxyl radicals which are free or esterified with a lower alcohol comprising from 1 to 6 carbon atoms, or an aromatic radical comprising from 6 to 10 carbon atoms.

Preferred salicylic acid derivatives of formula (I) are those in which R₁ represents a hydroxyl radical, R₂ represents a hydrogen atom, R₃ is in position 5 of the benzene ring, and R₅ represents a saturated aliphatic radical comprising from 3 to 15 carbon atoms.

When the composition comprises a salicylic acid derivative of formula (I), the derivative in question may be, in particular, 5-n-octanoylsalicylic acid, 5-n-decanoylsalicylic acid, 5-n-dodecanoylsalicylic acid, 5-n-octylsalicylic acid, 5-n-heptyloxysalicylic acid, 4-n-heptyloxysalicylic acid, 5-tert-octyl-salicylic acid, 3-tert-butyl-5-methylsalicylic acid, 3-tert-butyl-6-methylsalicylic acid, 3,5-diisopropyl-salicylic acid, 5-butoxysalicylic acid, 5-octyloxy-salicylic acid, 5-propanoylsalicylic acid, 5-n-hexa-decanoylsalicylic acid, 5-n-oleoylsalicylic acid, 5-benzoylsalicylic acid, their monovalent and divalent salts, and mixtures thereof.

When the composition comprises a salicylic acid derivative of formula (I), the derivative in question is preferably 5-n-octanoylsalicylic acid (INCI name: Capryloyl Salicylic acid).

The salicylic acid and derivatives thereof according to the invention may also be present in the form of salts, and more particularly in the form of salts obtained by salification with a base.

Bases capable of salifying salicylic acid and derivatives thereof according to the invention include inorganic bases such as alkali metal hydroxides (sodium hydroxide and potassium hydroxide) or ammonium hydroxides, or, more preferably, organic bases.

It is preferred to use amphoteric bases, in other words bases having both anionic and cationic functional groups.

The amphoteric bases may be primary, secondary, tertiary or cyclic organic amines, and more especially amino acids. Examples of amphoteric bases include glycine, lysine, arginine, taurine, histidine, alanine, valine, cysteine, trihydroxymethylaminomethane (TRISTA) and triethanolamine.

According to another, very particularly preferred embodiment, the composition comprises both salicylic acid and 5-n-octanoylsalicylic acid.

In one embodiment, the salicylic acid and/or at least one derivative thereof may be present in a composition according to the invention in a proportion of 1% to 5% by weight, preferably from 1.5% to 2.5% by weight, of salicylic acid and/or at least one derivative thereof, relative to the total weight of the composition.

Polyols:

A composition according to the invention comprises at least one polyol, comprising preferably 2 to 6 hydroxyl groups, and more preferably from 2 to 3 hydroxyl groups. It is of course also possible to use a mixture of such polyols.

The polyols have the general formula CₙH₂ₙ₊₂Oₙ.

The polyols which can be used according to the invention include, without limitation, glycols (diols), triols, and mixtures thereof.

In one particular embodiment of the invention, the polyol comprises 2 to 3 hydroxyl groups

The polyol may be selected, individually or as a mixture, from the following:

diols, such as propylene glycol, butylene glycol, hexylene glycol, pentylene glycol, caprylyl glycol, polyethylene glycol, with polypropylene glycol preferred;

triols, such as 1,2,4-butanetriol, 1,2,6-hexanetriol, trimethylolethane, trimethylolpropane, glycerol, with glycerol preferred;

tetraols, such as pentaerythritol (tetramethylol-methane), erythritol, diglycerol or ditrimethylol-propane;

pentols such as xylitol;

hexyls such as sorbitol and mannitol; or else dipentaerythritol or triglycerol.

The polyol is preferably selected from diols, triols and mixtures thereof.

Very preferably the polyol is selected from glycerol, propylene glycol and mixtures thereof.

In one embodiment, the polyol or mixture of polyols may be present in a composition according to the invention in a proportion of at least 5% by weight, preferably between 5% and 60% by weight, or else between 5% and 40%, more preferably between 5% and 20% by weight, and, for example, between 5% and 15% by weight, relative to the total weight of the composition.

The polyol or mixture of polyols may also be present in a composition according to the invention in a proportion greater than or equal to 10% by weight, relative to the total weight of the composition, for example in a proportion of from 10% to 35% by weight, relative to the total weight of the composition.

In one particular embodiment, the composition will comprise a mixture of glycerol and propylene glycol. The composition will advantageously comprise about 5% by weight of glycerol and about 5% by weight of propylene glycol, relative to the total weight of the composition.

Gelling Agents:

A composition according to the invention comprises at least one gelling agent.

The gelling agent is, in particular, hydrophilic.

Depending on the fluidity of the composition it is desired to obtain, one or more gelling agents may be incorporated into a composition of the invention.

A hydrophilic gelling agent (or thickener) is a gelling agent (or thickener) which is soluble or dispersible in water.

Hydrophilic gelling agents include, in particular, water-soluble or water-dispersible thickening polymers. These may in particular be selected from:
modified or unmodified carboxyvinyl polymers, such as the products sold under the Carbopol names (CTFA name: carbomer) by Goodrich;
homopolymers or copolymers of acrylic or methacrylic acid or their salts and their esters, and in particular the products sold under the names Versicol F® or Versicol K® by Allied Colloids, Ultrahold 8® by Ciba-Geigy, polyacrylates and polymethacrylates such as the products sold under the names Lubrajel and Norgel by Guardian or under the name Hispagel by Hispano Chimica, polyacrylic acids such as Synthalen K; Cosmedia SP (crosslinked sodium polyacrylate);
polyacrylamides;
copolymers of acrylic acid and of acrylamide, sold in the form of their sodium salt under the Reten® names by Hercules, sodium polymethacrylate, sold under the name Darvan No. 7® by Vanderbilt, and sodium salts of polyhydroxycarboxylic acids, sold under the name Hydagen F® by Henkel;
polymers and copolymers of 2-acrylamido-2-methylpropanesulphonic acid, optionally crosslinked and/or neutralized, such as poly(2-acrylamido-2-methyl-propanesulphonic acid) sold by Clariant under the name Hostacerin® AMPS (CTFA name: ammonium polyacryldimethyl-tauramide);
crosslinked anionic copolymers of acrylamide and of AMPS, in the form of a W/o emulsion, such as those sold under the name Sepigel 305 (CTFA name: Polyacrylamide/C13-14 Isoparaffin/Laureth-7) and under the name Simulgel 600 (CTFA name: Acrylamide/Sodium acryloyldimethyltaurate copolymer/Isohexadecane/Polysorbate 80) by Seppic;
polyacrylic acid/alkyl acrylate copolymers such as Pemulen;
polysaccharide biopolymers such as xanthan gum, guar gum, carob gum, acacia gum, scleroglucans, chitin and chitosan derivatives, carrageenans, gellans, alginates, celluloses such as microcrystalline cellulose, carboxymethylcellulose, hydroxymethyl-cellulose, hydroxyethylcellulose and hydroxypropyl-cellulose;
hydrophilic clays, hydrophilic fumed silica;
and mixtures thereof.

A hydrophilic clay is a clay which is able to swell in water; this clay swells in water and, following hydration, forms a colloidal dispersion.

Clays are products which are already known per se and are described in, for example, the work “Minéralogie des argiles, S. Caillère, S. Hénin, M. Rautureau, 2nd edition, 1982, Masson”. Clays are silicates comprising a cation which may be selected from calcium, magnesium, aluminium, sodium, potassium and lithium cations and mixtures thereof. Examples of such products include the clays from the smectite family such as montmorillonites, hectorites, bentonites, beidellites and saponites, and from the vermiculite family, the stevensite family and the chlorite family.

These clays may be natural or synthetic in origin.

Hydrophilic clays include the smectites such as the saponites, hectorites, montmorillonites, bentonites and beidellite.

Hydrophilic clays include synthetic hectorites (also called laponites) such as the products sold by Laporte under the name Laponite XLG, Laponite RD and Laponite RDS (these products are sodium and magnesium silicates, and in particular sodium, lithium and magnesium silicates); bentonites such as the product sold under the name Bentone HC by Rheox; magnesium and aluminium silicates, especially hydrated types, such as the products sold by Vanderbilt Company under the name Veegum ultra, Veegum HS, Veegum DGT, or else calcium silicates, and especially that in synthetic form sold by the company under the name Micro-cel C.

The hydrophilic fumed silicas may be obtained by high-temperature hydrolysis of a volatile silicon compound in an oxyhydrogen flame, to produce a finely divided silica. The hydrophilic silicas have a large number of silanol groups on their surfaces. Hydrophilic silicas of this kind are sold, for example, under the names Aerosil 130®, Aerosil 200®, Aerosil 255®, Aerosil 300® and Aerosil 380® by Degussa, Cab-O-Sil HS-5®, Cab-O-Sil EH-5®, Cab-O-Sil LM-130®, Cab-O-Sil MS-55® and Cab-O-Sil M-5® by Cabot.

The hydrophilic fumed silica preferably has a particle size which may be nanometric to micrometric, ranging for example, approximately, from 5 to 200 nm.

The gelling agent is preferably selected from modified or non-modified carboxyvinyl polymers, such as the products sold under the name Carbopol Ultrez-20 by the company Lubrizol (INCI: Acrylates/C10-30 Alkyl Acrylate Crosspolymer), or else under the name Synthalen K (INCI: Carbomer) by the company 3V, or else under the name Carbopol 981 Polymer by the company Lubrizol.

In one embodiment of the invention, a composition of the invention may comprise from 0.01% to 30% by weight of gelling agents, in particular from 0.1% to 5% by weight and more particularly from 0.1% to 2% by weight of gelling agents, relative to the total weight of the composition.

Formulations:

The cosmetic and/or dermatological composition according to the invention is preferably formulated as an aqueous gel or an aqueous-alcoholic gel.

Very preferably it will be an aqueous-alcoholic gel.

Alcohols:

When the cosmetic and/or dermatological composition according to the invention is in the form of an aqueous-alcoholic gel, it will comprise at least one alcohol. This may comprise a mixture of alcohols.

Any alcohol commonly used in the field of cosmetology may be used in the context of the present invention.

According to one embodiment of the invention, the alcohol is selected from ethanol, isopropanol, propanol, methanol, hexanol and mixtures thereof.

According to one very preferred embodiment, the alcohol in question will be ethanol.

According to one embodiment, the alcohol may be present in a composition according to the invention in a proportion of at least (greater than or equal to) 10% by weight, preferably between 10% by weight and 50% by weight, in particular between 20% by weight and 40% by weight, relative to the total weight of the composition.

Other Additives

The composition according to the invention may further comprise various adjuvants which are commonly used in the field of cosmetology, such as dyes, pigments, fragrances, preservatives, sunscreen agents, sequestrants, fat-soluble or water-soluble actives, and pH modifiers (acids or bases).

A person skilled in the art is aware of how to adjust the amounts in accordance with the desired effect.

The composition according to the invention may further comprise, in addition, fat-soluble or water-soluble actives and also antibacterial or antimicrobial and antisebhorreic actives. It may also comprise anti-inflammatory agents and/or calmatives.

Sebum-Regulating or Antiseborrhoeic Agents

By “sebum-regulating or antiseborrhoeic agents” are meant, in particular, agents capable of regulating the activity of the sebaceous glands.

They include, in particular, the following:
retinoic acid, benzoyl peroxide, sulphur, vitamin B6 (or pyridoxine), selenium chloride and sea fennel;
mixtures of extract of cinnamon, of tea and of octanoylglycine, such as Sepicontrol A5 TEA from Seppic;
the mixture of capryloyl glycine, sarcosine and extract of Cinnamomum zeylanicum sold in particular by SEPPIC under the trade name Sepicontrol® A5;
other zinc salts, in addition to zinc salicylate, such as zinc pyrrolidonecarboxylate (or zinc pidolate), zinc lactate, zinc aspartate, zinc carboxylate and zinc cysteate;
other copper salts, in addition to copper pidolate, such as copper sulphate or copper pyrrolidone-carboxylate;
extracts of plants of the species Arnica montana, Cinchona succirubra, Eugenia caryophyllata, Humulus lupulus, Hypericum perforatum, Mentha piperita, Rosmarinus officinalis, Salvia officinalis and Thymus vulgaris, all sold, for example, by Maruzen;
extracts of meadowsweet (Spiraea ulamaria), such as that sold under the name Sébonormine® by Silab;
extracts of Laminaria saccharina seaweed, such as that sold under the name Phlorogine® by Biotechmarine;
mixtures of extracts of burnet roots (Sanguisorba officinalis/Poterium officinale), of ginger rhizomes (Zingiber officinalis) and of cinnamon bark (Cinnamomum cassia), such as that sold under the name Sebustop® by Solabia;
extracts of linseed, such as that sold under the name Linumine® by Lucas Meyer;
Phellodendron extracts such as those sold under the name Phellodendron extract BG by Maruzen or Oubaku liquid B by Ichimaru Pharcos;
mixtures of argan oil, of extract of Serenoa serrulata (saw palmetto) and of sesame seed extract, such as that sold under the name Regu SEB® by Pentapharm;
mixtures of extracts of rosebay willow herb, of Terminalia chebula, of nasturtium and of bioavailable zinc (microalgae), such as that sold under the name Seborilys® by Greentech;
extracts of Pygeum africanum, such as that sold under the name Pygeum africanum sterolic lipid extract by Euromed;
extracts of Serenoa serrulata, such as those sold under the Viapure Sabal names by Actives International, or those sold by Euromed;
mixtures of extracts of plantain, of Berberis aquifolium and of sodium salicylate, such as that sold under the name Seboclear® by Rahn;
clove extract, such as that sold under the name Clove extract Powder by Maruzen;
argan oil, such as that sold under the name Lipofructyl® by Laboratoires Sérobiologiques;
lactic protein filtrates, such as that sold under the name Normaseb® by Sederma;
Laminaria seaweed extracts, such as that sold under the name Laminarghane® by Biotechmarine;
sugarcane extracts, such as that sold under the name Policasonol® by Sabinsa;
oligosaccharides of Laminaria digitata seaweed, such as that sold under the name Phycosaccharide AC by Codif;
sulphonated shale oil, such as that sold under the name Ichthyol Pale by Ichthyol;
meadowsweet extract (Spiraea ulmaria), such as that sold under the name Cytobiol Ulmaire by Libiol;
sebacic acid, particularly sold in the form of a sodium polyacrylate gel under the name Sebosoft by Sederma;
glucomannans extracted from konjac tuber and modified with alkylsulphonate chains, such as that sold under the name Biopol Beta by Arch Chemical;
extracts of Sophora angustifolia, such as those sold under the name Sophora powder or Sophora extract by Bioland;
extracts of Cinchona succirubra bark, such as that sold under the name Red bark HS by Alban Muller;
extracts of Quillaja saponaria, such as that sold under the name Panama wood HS by Alban Muller;
glycine grafted onto an undecylene chain, such as that sold under the name Lipacide UG OR by Seppic;
a mixture of oleanolic acid and nordihydro-guaiaretic acid, such as that sold in the form of a gel under the name AC.Net by Sederma;
trialkyl(C₁₂-C₁₃) citrate sold under the name Cosmacol® ECI by Sasol; trialkyl(C₁₄-C₁₅) citrate sold under the name Cosmacol® ECL by Sasol;
10-hydroxydecanoic acid, and especially mixtures of 10-hydroxydecanoic acid, sebacic acid and 1,10-decanediol, such as that sold under the name Acnacidol® BG by Vincience;
specific PPAR-γ activators, such as those described in application WO 2005/053632;
extracts of plants of the genus Silybum;
sapogenins or plant extracts comprising them, more particularly extracts of diosgenin-rich Dioscoreae; and
extracts of Eugenia caryophyllata comprising eugenol and eugenyl glucoside, and mixtures thereof.

Preferred sebum-regulating agents which can be used in accordance with the invention are as follows:
sea fennel;
mixtures of extract of cinnamon, of tea and of octanoylglycine, such as Sepicontrol A5 TEA from Seppic;
the mixture of capryloyl glycine, sarcosine and extract of Cinnamomum zeylanicum sold in particular by Seppic under the trade name Sepicontrol® A5;
other zinc salts, in addition to zinc salicylate, such as zinc pyrrolidonecarboxylate (or zinc pidolate), zinc lactate, zinc aspartate, zinc carboxylate and zinc cysteate;
other copper salts, in addition to copper pidolate, such as copper sulphate or copper pyrrolidone-carboxylate;
extracts of meadowsweet (Spiraea ulamaria), such as that sold under the name Sébonormine® by Silab;
extracts of Laminaria saccharina seaweed, such as that sold under the name Phlorogine® by Biotechmarine;
mixtures of extracts of burnet roots (Sanguisorba officinalis/Poterium officinale), of ginger rhizomes (Zingiber officinalis) and of cinnamon bark (Cinnamomum cassia), such as that sold under the name Sebustop® by Solabia;
sapogenins or plant extracts comprising them, more particularly extracts of diosgenin-rich Dioscoreae; and
   and mixtures thereof.

Antimicrobial Agents

By antimicrobial agents are meant agents which have effects on the specific flora of greasy skin, such as, for example, P. acnes.

These effects may be alternatively bactericidal effects, or anti-bacterial-adhesion effects (preventing and/or reducing the adhesion of microorganisms), or effects acting on the biofilm of the bacteria to prevent them from multiplying.

They include, in particular, the actives and preservatives with antimicrobial activity that are cited in application DE10324567, incorporated into the present invention by reference.

Mention may also be made of the following: a hop cone extract (HOP CO2-TO extract from Flavex), an extract of St John's Wort (St John's Wort CO2-TO extract from Flavex), asiatic acid, extracts of Scutellaria baicolensis roots such as in BMB-CF from Naturogin, piroctone olamine, citrollic acid, sperillic acid, ethylhexyl glycerine (Sensiva from Shulke), gluceryl caprylate/caprate (Capmul from Abitec), calcium sodium phosphosilicate such as Bioactive glass powder from Schott, Actysse premier BG from Schott, silicon oxides from Ciba, metashines (derivatives of silver), bearberry extracts such as Gatuline equalizing from Gattefossé, 10-hydroxy-2-decanoic acid such as Acnacidol P from Vincience, sodium ursolate, azelaic acid, diiodomethyl p-tolyl sulphone or Amical Flowable from Angus, Malachite from Maprecos, Zincare from Elementis GmbH, arlatone dioic from Unichema; ellagic acid; 2,4,4′-trichloro-2′-hydroxydiphenyl ether (or triclosan), 1-(3′-4′-dichlorophenyl)-3-(4′-chloro-phenyl)urea (or triclocarban), 3,4,4′-trichloro-carbanilide, 3′,4′,5′-trichlorosalicylanilide, phenoxy-ethanol, phenoxypropanol, phenoxyisopropanol, hexamidine isethionate, metronidazole and its salts, miconazole and its salts, itraconazole, terconazole, econazole, ketoconazole, saperconazole, fluconazole, clotrimazole, butoconazole, oxiconazole, sulfaconazole, sulconazole, terbinafine, ciclopirox, ciclopiroxol-amine, undecylenic acid and its salts, benzoyl peroxide, 3-hydroxybenzoic acid, 4-hydroxybenzoic acid, phytic acid, N-acetyl-L-cysteine, lipoic acid, azelaic acid and its salts, arachidonic acid, resorcinol, octopirox or piroctone olamine, octoxyglycerine or octoglycerine, octanoylglycine (Lipacid C8G® from Seppic), caprylyl glycol, 10-hydroxy-2-decanoic acid, dichlorophenyl imidazole dioxolane and its derivatives described in patent application WO9318743, zinc derivatives and in particular zinc pidolate (Zincidone from Solabia), copper derivatives, salicylic acid and other derivatives of salicylic acid, iodopropynyl butylcarbamate, 3,7,11-trimethyldodeca-2,5,10-trienol or farnesol, phytosphingosines; Sepicontrol® from Seppic, an argan extract such as Argapure LS9710®, Sebosoft® from Sederma, quaternary ammonium salts such as cetyltrimethylammonium salts, cetylpyridinium salts, ethanol, etc. and mixtures thereof.

As agents that prevent and/or reduce the adhesion of microorganisms, mention may especially be made of the following: phytantriol and its derivatives as described in patent application EP 1 529 523, plant oils such as wheat germ oil, calendula oil, castor oil, olive oil, avocado oil, sweet almond oil, peanut oil, jojoba oil, sesame oil, apricot kernel oil, sunflower oil, and macadamia oil, which are described in patent EP 1 133 979, or else other fatty substances such as disodium cocoamphodiacetate, oxyethylenated (7 EO) glyceryl cocoate, Poloxamers, hexadecenyl succinate 18, octoxyglyceryl palmitate, octoxyglyceryl behenate, dioctyl adipate, PPG-15 stearyl ether, and branched C₁₂-C₁₃ dialcohol tartrate described in patent EP 1 129 694.

In particular, as regards the propagation of P. acnes, mention may be made of pentylene glycol, nylon-66 (polyamide 66 fibres), rice bran oil, Celvol 540 PV alcohol (polyvinyl alcohol), Akorex L from Karlshamns, and fructose derivatives.

Mention may also be made of certain surfactants having an antimicrobial effect such as sodium cocoamphoacetate or disodium cocoamphodiacetate such as Miranol C2M CONC NP, betaines such as Genagen KB cocoyl betaine from Clariant, sodium lauryl ether sulphate such as Emal 270 D from Kao, decyl glucoside such as Plantacare 2000 UP, branched C₁₂-C₁₃ dialcohol malates such as Cosmacol EMI, propylene glycol monoesters such as propylene glycol monolaurate, monocaprylate or monocaprate, sodium lauroyl oat amino acid such as Proteol OAT, lauryl dimethylamine betaine such as Empigen BB/LS and also polyquaternary ammonium compounds such as Quaternium-24 or Bardac 2050 from Lonza and those described in L'Oréal patent FR 0 108 283.

As preferred antimicrobial agents, an agent will be used in the compositions of the invention that is selected from caprylyl glycol, zinc derivatives including zinc pidolate (Zincidone® from Solabia), copper derivatives, octoglycerine or octoxyglycerine, 10-hydroxy-2-decanoic acid, and mixtures thereof.

Calmatives

Examples of “calmatives” that can be used in the compositions of the invention include the following:
vitamins, such as vitamin B3 or provitamin B5;
allantoin;
betaglycyrrhetinic acid, extracts comprising it, such as, for example, extract of Glycyrrhiza glabra (liquorice), and complexes comprising it, such as allantoin/glycyrrhetinic acid complex;
lyophilized or non-lyophilized planktons, extracts thereof and complexes thereof;
escin and plant extracts comprising it, such as extract of horse chestnut;
xanthine derivatives, such as diethylaminoethyltheophylline hydrochloride;
waters and extracts (for example aqueous, aqueous-alcoholic or aqueous-glycolic extracts) of flowers and plants, such as cornflower water, camomile water, mint water, lime water, rose water, extracts of Rosaceae (e.g. Rosa gallica), extracts of peony, extracts of hawthorn, extracts of yarrow, extracts of mallow, extracts of marigold, extracts of sweet clover, extracts of sage, elderberry water, extracts of Ginkgo biloba, extracts of arnica, extracts of oregano, extracts of green tea, extracts of water lily blossom, extracts of iris, extracts of birch bark, extracts of Aloe vera and extracts of mint leaves, such as Calmiskin GR from Silab;
asiatic acid and plant extracts comprising it, such as Centella asiatica;
bisabolol;
fruit extracts, such as extract of pineapple, extract of papaya, and of guava;
algae, especially those of the type Laminaria (for example red or brown algae), such as the extract of brown alga Padina pavonica, such as HPS 3 Padina pavonica sold by the company Alban Muller;
pyrrolidonecarboxylates, and especially those of zinc (Zn-PCA) or of copper (Cu-PCA);
oils of plant origin, such as canola seed oil and shea oil;
essential oils, for example coriander oil, balm oil, lavender oil, mint oil, camomile oil and mixtures thereof;
acexamic acid and transexamic acid (4-trans-aminomethylcyclohexanecarboxylic acid);
ursolic acid and extracts comprising it, such as extract of rosemary leaf;
polysaccharides containing fucose, such as Fucogel 1000, sold by Solabia (aqueous solution comprising 1% polysaccharide solids comprising fucose, galactose and galacturonic acid);
electrolytes, and more particularly an aqueous mixture comprising from 30% to 35% of magnesium chloride, from 20% to 28% of potassium chloride, from 3% to 10% of sodium chloride, from 0.2% to 1% of calcium chloride, from 0.1% to 6% of magnesium bromide and from 0.1% to 0.5% of insolubles, the said mixture here being called “Dead Sea Bath Salts”, since it corresponds to the major salts present in the Dead Sea;
galactolipids, for example those obtained from oats, such as, for example digalactosyl diglyceride or monogalactosyl diglyceride.

The packaging assembly according to the invention permits application of a cosmetic and/or dermatological composition according to the invention to the skin, especially the skin of the face and/or body.

The present invention further provides a cosmetic method of caring for the skin, especially for greasy and/or acne-affected and/or acne-prone and/or seborrhoeic skin, comprising the use, on the surface of the skin, of an assembly for packaging and application of a cosmetic and/or dermatological composition according to the invention.

The present invention additionally provides for the cosmetic use of an assembly for packaging and application of a cosmetic and/or dermatological composition according to the invention for caring for greasy skin and/or for skin exhibiting imperfections, especially having blackheads.

The invention compositions and combinations are preferably used by human subjects desirous of the benefits noted herein, subjects “in need of” these benefits. Such subjects are typically suffering from one or more of the conditions, symptoms, etc. addressed by the present invention, such as by self diagnosis or cosmetician or medical diagnosis, or are at recognized and appreciated risk of developing such conditions, etc. and who intentionally use the invention methods, compositions and combinations to treat, address, combat, prevent, etc. the effects of such conditions, etc. The application also clearly describes and supports the simple application of the invention composition on the skin and its integuments regardless of any purpose or intent.

The above written description of the invention provides a manner and process of making and using it such that any person skilled in this art is enabled to make and use the same, this enablement being provided in particular for the subject matter of the appended claims, which make up a part of the original description.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The invention will now be described with reference to the examples below, which are given for illustration and not in limitation.

### EXAMPLES

### Example 1

### Compositions

### Example 1(a)

The composition below represents a composition in the form of an aqueous-alcoholic gel having a viscosity at 25° C. of 1 poise. This composition is subsequently packaged in a cosmetic and/or dermatological packaging and application assembly according to the invention.

| Phase A1: | |
|---|---|
| Water | qs 100% |
| Glycerol | 5% |
| Plant extract | 0.5% |
| Zinc gluconate | 0.1% |
| Phase A2: | |
| Carbopol* | 1.2% |
| Phase B1: | |
| Propylene glycol | 5% |
| 5-n-Octanoylsalicylic acid | 0.2% |
| Salicylic acid | 2% |
| Phase B2: | |
| Ethanol | 35% |
| Phase B3: | |
| Fragrance | 0.2% |
| Phase C: | |
| Water | 5% |
| Neutralizing agent | 0.4% |

*gelling agent

Procedure:

Preparation of phase C: the neutralizing agent is dissolved in cold water.

Preparation of phase B1: form a paste of the 5-n-octanoylsalicylic acid and the salicylic acid in propylene glycol.

Using a cold deflocculating device, mix the actives of phase A1 to full homogenization (clear brown liquid) and then, in a vortex, incorporate the carbopol of phase A2, and then mix until a smooth gel is obtained.

Subsequently, at ambient temperature and with stirring, add phase B1, and then phase B2.

Subsequently add the fragrance.

Subsequently add phase C to neutralize the gel, with stirring.

### Example 1(b)

Composition identical to that of Example 1(a), the only difference being the removal of 5-n-octanoyl-salicylic acid and its replacement by an equivalent amount of water.

### Example 2

### Comparative Tests

The composition according to Example 1(a), packaged in a cosmetic and/or dermatological packaging and application assembly according to the invention, comprising a metal (stainless steel) applicator ball, is compared to a composition (packaged in strictly identical packaging) whose only difference is the absence of 5% of propylene glycol, thereby taking the total polyol content to 5% as against 10% in the composition according to Example 1(a).

When the ball had been first initiated in the course of a first application, it was observed that the composition containing only 5% of polyol gave rise to recrystallization of the salicylic acid after two months of storage at temperature (45° C.) on the surface of the ball. This phenomenon is characterized by the presence of small, disparate crystals on the surface of the ball, but also in the area of contact between the ball and its support (thereby giving rise to a blockage and a quality defect).

The level of polyol(s) present in the composition according to Example 1(a) allowed this problem to be avoided, hence producing easy rolling of the ball on itself.

1. Assembly (1) for packaging and application of a cosmetic and/or dermatological composition, comprising a container (2) having an opening (3), an applicator element (4), mounted in such a way as to be freely rotatable, in the vicinity of said opening (3), the applicator element (4) comprising an outer surface which can be brought into fluidic communication with the inside of the container (2), wherein the container (2) comprises a cosmetic and/or dermatological composition in the form of a gel having a viscosity at 25° C. of between 0.1 and 60 poises, and comprising, in a physiologically acceptable medium:
(a) at least 1% by weight of salicylic acid and/or at least one derivative thereof, relative to the total weight of the composition;
(b) at least one polyol; and
(c) at least one gelling agent.

2. Assembly (1) for packaging and application of a cosmetic and/or dermatological composition according to claim 1, wherein the applicator element (4) is a metal ball.

3. Assembly (1) for packaging and application of a cosmetic and/or dermatological composition according to claim 1, wherein said cosmetic and/or dermatological composition in the form of a gel has a viscosity at 25° C. of between 0.5 and 10 poises.

4. Assembly (1) for packaging and application of a cosmetic and/or dermatological composition according to claim 1, wherein said cosmetic and/or dermatological composition comprises from 1% to 5% by weight of salicylic acid and/or at least one derivative thereof relative to the total weight of the composition.

5. Assembly (1) for packaging and application of a cosmetic and/or dermatological composition according to claim 1, wherein said cosmetic and/or dermatological composition comprises at least 5% by weight of polyol(s) relative to the total weight of the composition.

6. Assembly (1) for packaging and application of a cosmetic and/or dermatological composition according to claim 1, wherein the polyol comprises 2 to 3 hydroxyl groups.

7. Assembly (1) for packaging and application of a cosmetic and/or dermatological composition according to claim 6, wherein the polyol is selected from the group consisting of glycerol, propylene glycol and mixtures thereof.

8. Assembly (1) for packaging and application of a cosmetic and/or dermatological composition according to claim 1, wherein said cosmetic and/or dermatological composition is in the form of an aqueous-alcoholic gel comprising at least one alcohol.

9. Assembly (1) for packaging and application of a cosmetic and/or dermatological composition according to claim 8, wherein said cosmetic and/or dermatological composition comprises at least 10% by weight of alcohol(s) relative to the total weight of the composition.

10. Assembly (1) for packaging and application of a cosmetic and/or dermatological composition according to claim 8, wherein the at least one alcohol is selected from the group consisting of ethanol, isopropanol, propanol, methanol, hexanol and mixtures thereof.

11. Assembly (1) for packaging and application of a cosmetic and/or dermatological composition according to claim 10, wherein the at least one alcohol is ethanol.

12. Assembly (1) for packaging and application of a cosmetic and/or dermatological composition according to claim 1, wherein said cosmetic and/or dermatological composition comprises from 0.01% to 30% by weight of gelling agent(s) relative to the total weight of the composition.

13. A method of treating greasy and/or acne-affected and/or acne-prone and/or seborrhoeic skin, comprising applying the assembly (1) for packaging and application of a cosmetic and/or dermatological composition according to claim 1 to the skin to apply the cosmetic and/or dermatological composition in the form of a gel having a viscosity at 25° C. of between 0.1 and 60 poises to the skin to treat the skin.

14. A method of treating blackheads comprising applying the assembly (1) for packaging and application of a cosmetic and/or dermatological composition according to claim 1 to the skin to apply the cosmetic and/or dermatological composition in the form of a gel having a viscosity at 25° C. of between 0.1 and 60 poises to the skin to treat the skin.
